(19) United States
(12) Patent Application Publication
(10) Pub. No.: US 2024/0198069 A1
(43) Pub. Date: Jun. 20, 2024

(54) OZONE THERAPEUTIC TREATMENT APPARATUS

(71) Applicant: Spence Spencer, Scottsdale, AZ (US)

(72) Inventor: Spence Spencer, Scottsdale, AZ (US)

(21) Appl. No.: 18/589,419

(22) Filed: Feb. 27, 2024

Related U.S. Application Data
(63) Continuation of application No. PCT/US23/25684, filed on Jun. 19, 2023.
(60) Provisional application No. 63/353,064, filed on Jun. 17, 2022.

(51) Int. Cl. A61M 37/00; A61H 33/06; A61K 33/00

(52) U.S. Cl. CPC A61M 37/00; A61H 33/066; A61K 33/00; A61H 2033/068; A61H 2201/105; A61M 2037/0007; A61M 2202/0216; A61M 2205/054; A61M 2210/04

(57) ABSTRACT

An ozone therapeutic treatment apparatus utilizes magnetism to move the ozone to or across a treatment condition. The treatment condition may be an internal treatment condition and the ozone is implanted into the body by injection or by absorption through the skin. Ozone is diamagnetic, because all of its electrons are paired. The ozone implanted in the body is drawn through the body, across a treatment condition, toward a magnet. The magnet may be a permanent magnet that is placed on or near the skin or may be an electromagnet, wherein the magnetic field is varied as a function of time. The ozone therapeutic treatment apparatus may be used to treat a wide range of conditions including scar tissue in joints, tumors and herpes.

### CROSS REFERENCE TO RELATED APPLICATIONS

This application is a continuation in part of International application No. PCT/US2023/025684, filed on Jun. 19, 2023, which claims the benefit of priority to U.S. provisional patent application No. 63/353,064, filed on Jun. 17, 2022; the entirety of which is hereby incorporated by reference herein; the entirety of all prior applications are hereby incorporated by reference herein.

### BACKGROUND OF THE INVENTION

### Field of the Invention

The invention relates to an ozone therapeutic treatment apparatus and method of treatment using same.

### Background

Ozone is used to treat a number of conditions by introduction into the body. Symptoms from scar tissue in joints can be reduce by the application of ozone to the joint, such as by injection of ozone. Injecting ozone deep into the joint can be painful and provides a limited quantity of ozone for treatment.

### SUMMARY OF THE INVENTION

The invention is directed to an ozone therapeutic treatment apparatus and method of treating a treatment condition or location in a living body by using magnetism to move ozone to said treatment location as well as control/direct the healing potential towards the desired location. In an exemplary embodiment, the treatment condition is an external treatment condition, a treatment location on the exterior of the living body, such as on the skin, on the head and the like. In an exemplary embodiment, the treatment condition is an internal treatment condition, a treatment location within a living organism, and the ozone is implanted into the body by injection or by absorption through the skin. Ozone is diamagnetic, because all of its electrons are paired and therefore is subject to magnetic forces. The ozone is introduced into the body and directed or drawn through the body, across or to a treatment location, toward a magnet. The magnet may be a permanent magnet, may be a switchable magnet, wherein the magnetic polarity is switched back and forth, or may be an electromagnet, wherein the magnetic field is varied as a function of time. A magnet may be a porous magnet having one or more apertures through the magnet from a skin contact surface of the magnet to an opposing surface. The apertures through the magnet allows the flow of gases and in particular ozone through the magnet. An ozone therapeutic treatment apparatus may utilize one or more magnetic ozone interfaces that incorporates a porous magnet and a pressure apparatus to generate a flow of ozone through the porous magnet and into the living body or to the skin of the living body. In an exemplary embodiment, ozone is forced into the body through a first magnetic ozone interface and drawn to or across a treatment location by a second magnetic ozone interface.

Ozone is a diamagnetic substance that is characterized by paired electrons, e.g., no unpaired electrons. According to the Pauli Exclusion Principle which states that no two electrons may occupy the same quantum state at the same time, the electron spins are oriented in opposite directions. The ozone used in the ozone therapeutic treatment apparatus may be an ozone rich gas which has 5% or less mole fraction of other gases, or 3% or less, 1% or less, or even 0.5% or less mole fraction of other gasses.

Ozone may be in various forms such as O₃ or O₈, for example. The concentration of ozone may be in the gamma range, such as 1-150 gamma, wherein: 1 mg/l ozone in air=1 g/m3=1 ug/ml=1 gamma.

An ozone introducer is configured to implant ozone into a body and may comprise an ozone cup that is configured to be placed on the skin of the body to form a seal that retains said ozone against the skin for absorption through the skin. The cup may contain ozone that is pressurized, such as about 10 kPa or more, about 25 kPa or more, about 50 kPa or more, about 100 kPa or more, about 250 kPa or more and any range between and including the pressure values provided. The ozone cup may be secured to the skin by a strap, clamp or other means to produce an effective seal on the skin. The ozone cup may be coupled with an ozone source to provide a continuous supply of ozone to the ozone cup as the ozone is absorbed through the skin. An ozone generator may be used to produce ozone and a hose may extend from the generator to the ozone cup to provide a supply of ozone to the ozone cup. An ozone source may provide or an exemplary ozone generator may be configured to produce ozone in a particular concentration such as about 50% of more, 90% or more, 95% or more, or even 100% by molar concentration, or the concentration may be 10 ppm or more, about 50 ppm or more, about 100 ppm or more, about 200 ppm or more about 500 ppm or more, about 1000 ppm or more, and any range between and including the ozone concentrations provided. A high concentration of ozone may be about 80% or more by molar concentration. A porous magnet may be configured within the ozone cup and allow ozone to flow through the porous magnet into or out of the skin of the patient.

An ozone introducer may comprise an injector that has a needle configured to puncture through the skin. The needle is hollow and has an opening proximal the extended end to allow the ozone to flow through the needle and into the body. The needle may be coupled with a hose and an ozone source to provide a continuous stream of ozone into the body, referred to herein as continuous injection. The needle may be coupled with a syringe having an internal receptacle and a plunger to force the ozone within the internal receptacle out of the syringe through the needle. The plunger may be configured for manual operation.

An ozone introducer may be an ozone jet injector that comprises a pressurizing device configured to pressurize the ozone and introduce the ozone into the living body under high pressure. A jet injector is a type of medical injecting syringe used for ozone jet injection. A high-pressure stream of ozone in either gas or dissolved in a liquid, penetrates the living body, such as the skin to deliver ozone to a targeted treatment location, such as the epidermis, muscle, into a joint or bone, or organ, for example. A pressurizing device may be a piston in an enclosed liquid-filled chamber, coupled with the injecting syringe. The piston is usually pushed by the release of a compressed metal spring.

Ozone may be injected into or introduced intravenously such as by injection with a syringe or through an intravenous bag having ozone dissolved in the intravenous solution. The ozone may then be directed to a particular treatment location by use of a magnet placed on the living body, such as near an organ. The ozone therapeutic treatment apparatus may utilize ozonated blood that is drawn from the patient, ozonated and then delivered back to the patient for treatment. The ozonated blood may be injected into the patient proximal to a treatment condition or may be simply circulated back into the vascular system to treat blood related illness or disease or conditions including autoimmune diseases. The ozone may be introduced directly intravenously or may be configured in a solution, such as a saline solution.

Ozone may be introduced through an enclosure, such as a sauna that heats the person or a portion of the person's body and has an enclosure for retaining a concentration of ozone. The enclosure may fit around a portion of a person's body, such as an arm or leg and may be a sleeve that seals around the person's body part to retain an elevated concentration of ozone within the interior, as described herein, and may have a heating element to heat the person's body (living body) or body part to aid in the introduction of ozone into the person's body. In an exemplary embodiment, the sauna is large enough for the person to fit within the sauna and a breathing apparatus may be used to provide breathing air to the patient or the sauna may have an opening to allow the person's head to extend out of the sauna for safe breathing of air without elevated concentrations of ozone. The use of heat opens the pores and allows the ozone to more effectively penetrate into the patient's skin. The sauna may heat the person of body part of the person to about 30° C. or more, about 40° C. or more, about 45° C. or more, about 50° C. or more and any range between and including the temperatures provided. An ozone generator may provide a concentration of ozone into the interior of the enclosure or heated sauna according the concentrations provided herein. The concentration of ozone within the interior of the enclosure or heated sauna may be elevated and have a concentration such as about 50% of more, 90% or more, 95% or more, or even 100% by molar concentration, or the concentration may be 10 ppm or more, about 50 ppm or more, about 100 ppm or more, about 200 ppm or more about 500 ppm or more, about 1000 ppm or more, and any range between and including the ozone concentrations provided. The ozone may be included with steam or heated water vapor to aid in absorption of the ozone into the skin. One or more magnets as described herein may be configured within the enclosure of the sauna and placed against or near the skin to draw ozone to or across a treatment location.

In addition, an enclosure for a body part, such as a limb or for a body of person with an aperture for their head, may be pressurized, wherein the ozone at an elevated pressure is introduced into the chamber for improved absorption. The pressure may be about 1.25 atmospheric pressure (atm), or about 1.5 atm or more, about 1.75 atm or more, about 2.0 atm or more and any range between and including the pressures provided. The pressure may be pulsed to further aid in driving the ozone into a person's body, such as through absorption through the skin. The chamber may extend around a limb and have pressurized gaskets around and end or ends to seal the chamber around a limb. A chamber may be a body chamber for the person's full body and may have an aperture for the person's head to extend out of the chamber. Alternatively, a full body chamber may require a breathing apparatus, such as a conduit that extends outside of the chamber. The ozone within the living body may be directed by a magnet that is placed on or near the skin to produce a magnetic force to draw the internal ozone within the body, toward the magnet. The magnet may be a permanent magnet, switchable magnet or an electro magnet. The magnet may produce a magnetic field having a strength of about 500 Gauss or more, about 1,000 Gauss or more, about 2,000 Gauss or more, about 4000 Gauss or more, about 6,000 Gauss or more, about 10,000 Gauss or more, about 15,000 Gauss or more and any range between and including the values provided. A magnet that produces a 4,000 Gauss surface strength will have a penetration of about three inches into the skin, as an example. Also, a magnetic field strength of about 15,000 is approximately the magnetic field strength used in a resonance imaging system (MRI) or is equivalent to the strength of a coin-sized magnet that can lift 9 kg. An exemplary magnet may be a permanent or switchable magnet. A pulsed electro-magnetic field (PEMF) may not be preferred however as this may denature the ozone. A static magnetic field, may be preferred for this reason. A magnet may be sized to produce an effective magnetic field to pull ozone to a treatment location and may be planar in shape and have an in plane dimension, length or width or diameter of about 25 mm or more, about 50 mm or more, about 75 mm or more, about 100 mm or more and any range between and including the dimensions provided.

A magnet may be a porous magnet that has apertures through the magnet from a skin contact surface to a cup surface. The porous magnet may be porous metal, or having apertures through the magnet. A magnet may be a ring magnet having an aperture, such as a circular aperture, surrounded by the magnet material. A ring magnet may be attached to an ozone cup or ozone cup assembly having an inner ozone cup surrounded by a collection cup to capture any ozone that may leak from the internal ozone cup. A magnet may be flexible and may be configured to extend around a person limb, such as by wrapping around their limb. This flexible type of magnet may be useful in a magnetic ozone interface that combines the magnet and the ozone cup into a wrap that can be configured around a limb of a person, arm, foot, hand, or over a joint, such as over the shoulder or around the hip, for example. A large magnetic ozone interface may be wrapped around a torso of a person and may have a plurality of magnets for locating near a treatment location and/or a flexible magnet that wraps around the curvature of the body.

A magnet may be a switchable magnet, a magnet that can be cycled on and off without electricity. A switchable magnet is made from two blocks of iron, with a round cavity bored through the center. The halves are joined together with a non-ferrous material such as brass or aluminum. A round permanent magnet is inserted into the bored hole configured between the two halves and rotation of the permanent magnet turns on the blocks of iron as a magnet. Rotation of the permanent magnet changes the orientation of the magnetic field and can therefore turn the magnet on and off.

An electromagnet is a magnet having a soft metal core that is made into a magnet by the passage of electric current through a coil surrounding the magnet. The magnetic field strength may be varied from neutral to a positive value. An electromagnet produces a magnetic field that can be quickly changed by controlling the amount of electric current in the winding. This change in magnetic field strength may be advantageous to moving ozone through the body and into and through a treatment condition.

An exemplary magnetic ozone interface may combine an ozone cup with a magnet for application to a treatment location. A magnet may have apertures for allowing ozone to pass therethrough and an ozone cup may be coupled to the magnet. An ozone cup may be part of an ozone cup assembly having an internal ozone cup surrounded by a collection cup. The internal ozone cup receives ozone from an ozone source that may force ozone into the interior of the ozone cup with pressure. The ozone source may be an ozone generator and may produce ozone with a concentration of ozone as described herein. The collection cup is configured around the ozone cup and configured to capture any ozone that leaks from the interior ozone cup. The collection cup may be coupled with an ozone destruction device that may create a vacuum pressure to draw the ozone to the ozone destruction device from the collection cup. The collection cup may have a material that reacts with ozone to destroy the ozone, such as magnesium dioxide MnO2, a catalyst used in ozone destruction. The interior of the collection cup may be coated with magnesium dioxide, for example.

An exemplary magnetic ozone interface may be configured to be wrapped around a person limb, such as the arm, hand, foot or their leg, and may include an ozone cup assembly with an internal ozone cup and collection cup configured around the collection cup. An exemplary magnetic ozone interface may be configured to wrap around a body part including over the shoulder, head, around the back or hip of a person or animal and the like.

The ozone therapeutic treatment apparatus may further use heat for heating the treatment location to increase blood flow and/or to open pores in the skin for higher absorption rates of ozone. The ozone cup may be heated and/or the flow of gas, including ozone, into the cup may be heated to increase ozone absorption.

The ozone therapeutic treatment apparatus may further use light, such as red light or lasers in combination with the ozone treatment to stimulate healing, especially for skin conditions. A light element, such as a red light emitter or laser may be incorporated into an ozone cup and direct light onto the skin of the patient. The light may be on prior to, during and/or after the treatment with ozone.

The ozone therapeutic treatment apparatus may be used to treat a wide variety of treatment conditions including skin or dermatological conditions, vascular conditions, autoimmune conditions, orthopedic conditions, oncological conditions, disease conditions, infections, digestive conditions, psychological conditions, hair loss and the like.

Skin or dermatological conditions include but are not limited to, scars acne, atopic dermatitis, epidermolysis bullosa, eczema, hidradenitis suppurativa, ichthyosis, pachyonychia congenita, pemphigus, psoriasis, Raynaud's phenomenon, rosacea, scleroderma, vitiligo, fever blisters, shingles, spider veins and the like.

The ozone therapeutic treatment apparatus may be used to treat hair loss and the ozone cup may be placed over the scalp to provide a flow of ozone to the scalp. A porous magnet of a magnetic ozone interface may drive the ozone into the scalp to stimulate hair follicles to grow. Likewise, the ozone therapeutic treatment apparatus may be used to treat various dermatological conditions, wherein the ozone cup is placed over a skin treatment location and a flow of ozone rich gas is supplied. Again, the ozone may flow through a porous magnet to further drive the ozone into the skin, such as into deeper layers of the epidermis, dermis or hypodermis. In addition, additional ozone may be injected into the skin proximal to the ozone cup, such as around the perimeter of the ozone cup to produce an enriched ozone concentration in the skin. Injected ozone may be injected deeper into the epidermis, dermis or hypodermis. Some skin conditions that may be treated include, but are not limited to: acne, atopic dermatitis, epidermolysis bullosa, eczema, hidradenitis suppurativa, ichthyosis, pachyonychia congenita, pemphigus, psoriasis, Raynaud's phenomenon, rosacea, scleroderma, vitiligo, fever blisters, shingles, spider veins and the like. Also, the ozone therapeutic treatment apparatus may be used to treat wrinkles and age spots and may be used to reduce cellulite. The ozone therapeutic treatment apparatus may be used to reduce cellulite by application to the skin.

The ozone therapeutic treatment apparatus may be used to treat endometriosis and may be placed on either side of the abdomen or on the front of the abdomen and on the back to force ozone through the endometriosis site.

The ozone therapeutic treatment apparatus may be used to treat lyme disease by driving ozone into the brain or into an affected area such as a joint. Treatment of the brain may aid with Alzheimer's, brain fog and the like. In the method with the ozone cup on the head, ozone crosses the blood brain barrier and is effective at dissolving the biofilm and aiding to eliminate the pathogen. Other conditions that may be treated by directing ozone into the brain include, Alzheimer's, and Parkinson's.

The ozone therapeutic treatment apparatus may be used to treat Myofascial pain syndrome, which is a chronic condition that causes pain in the musculoskeletal system. Once the defect is located the ozone cup or multiple cups (or bags) are placed on the affected area with a magnet or magnets on the opposite side to drive ozone through the affected area. The magnets may be configured on opposing or opposite side of the ozone cup, wherein ozone is introduced into the body and Heat can be added to improve the transdermal absorption. Various light waves may be used to charge the ozone/ozonides to increase the strength of the ozone. This same method may be used to treat soft tissue injuries to muscles, tendons and ligaments and to reduce joint inflammation. The method may be used to treat inflammatory arthropathies, or arthritis, osteoarthritis and/or post traumatic arthritis.

One or more magnets may be used with an exemplary ozone therapeutic treatment apparatus to draw ozone through tissue toward the magnet(s). The magnets may be configured on opposite sides of a location of ozone introduction into a person's body, such as on opposing or opposite sides of a limb, such as an arm or leg with ozone introduced between the magnets.

An exemplary magnet may be a permanent magnet or an electromagnet that is powered by an electromagnet generator that may enable control of the frequency and amplitude of the magnetic field. A oscillation of the magnetic field or field strength may increase the draw of ozone through the body, such as through tissue. The magnets may be porous magnets to allow for the flow of gas, such as ozone therethrough.

The ozone therapeutic treatment apparatus may be used to treat cervical stenosis, wherein bone spurs form causing cervical spinal stenosis. Ozone cupping with magnets dissolves the defects while improving the cellular health of the neck.

The ozone therapeutic treatment apparatus may be used to treat infections including bacterial infections. The ozone may be driven and directed toward a location of infection, such as due to a surgical procedure, or a gall bladder rupture and the like.

The ozone therapeutic treatment apparatus may be used to treat Carpal Tunnel Syndrome, and calm the inflammation by dissolving the hardened inflamed tissue.

The ozone therapeutic treatment apparatus may be used to treat Fibromyalgia and dissolve biofilm thereby calming inflammation.

The ozone therapeutic treatment apparatus may be used to treat vascular conditions including, but not limited to heart attack, stroke, heart arrhythmia by drawing ozone through the heart muscle and tissue to clean away scar tissue and calm inflation. The method may reduce cholesterol deposits or plaque in the heart or in other areas of the vascular system. The method may be used to reduce blood pressure by clearing away blockages in the vascular system.

The ozone therapeutic treatment apparatus may be used to treat diabetic ulcers by driving ozone across or into the ulcer. Healing light frequency may be added to charge the cellular activity to further promote healing.

The ozone therapeutic treatment apparatus may be used to treat lipomas, a non-cancerous (benign) lump that forms due to an overgrowth of fat cells.

The ozone therapeutic treatment apparatus may be used to treat oncological conditions, including tumors, such as cancerous tumors by directing ozone onto and into the tumor to reduce the size and eliminate the tumor. Skin cancer, precancerous skin growths, squamous cell carcinomas, any cancerous tumor or growth. Ozone may be introduced into or onto the living body as described herein and further directed to the treatment location by placement of a magnet on the living body.

The ozone therapeutic treatment apparatus may be used to treat internal organs that are diseased or are not functioning properly, such as the liver, kidney, bladder, stomach, thymus, glands, intestine (large and small), lymph nodes, testicles, adrenal glands, heart, lung, ovary, brain, bones, fallopian tubes, pancreas, spleen, uterus, gallbladder, esophagus, ear and mouth. A magnet may be placed on or near the organ, preferably externally to the body and ozone may be introduced by a needle injection, through absorption through the skin, in the blood or any combination thereof.

The ozone therapeutic treatment apparatus may be used to treat autoimmune disorders, such as Lupus, AIDS, Addison disease, Celiac disease—sprue (gluten-sensitive enteropathy), Dermatomyositis, Graves disease, Hashimoto thyroiditis, Multiple sclerosis, Myasthenia gravis, Pernicious anemia, Reactive arthritis, Rheumatoid arthritis, Sjögren syndrome, Systemic lupus erythematosus, and Type I diabetes. A magnet may be placed on or near the brain and ozone may be introduced by a needle injection, through absorption through the skin, in the blood or any combination thereof.

The ozone therapeutic treatment apparatus may be used to treat the vasculature system and may be used to open up arteries to prevent blockage or blood clots, soften hardened arteries, and clean the arteries by the application of ozone and a magnet near the affected locations. Ozone may be introduced into the bloodstream or injected into the body near the affected vascular treatment location.

Treatment to the brain may improve chronic fatigue, attention deficit disorder (ADD) or attention deficit hyperactivity disorder, (ADHD) or symptoms related to Lyme disease, by application to the brain. Also, an affected joint wherein Lyme disease has caused inflation may be treated as described herein.

The ozone therapeutic treatment apparatus may be used to treat anxiety, stress, and/or depression, headaches, migraines, irritability, mood swings and chronic fatigue by application to the head. Also, treatment of the head with ozone and magnets may improve memory or prevent memory loss. The methods described herein may slow or prevent dementia or Alzheimer's.

The ozone therapeutic treatment apparatus may be used to treat conditions or disease of the digestive system, such as constipation, yeast infection, bladder infection, urinary tract infections, diarrhea, ulcers, hemorrhoids, gastroesophageal reflex disease, or reflux and the like.

The ozone therapeutic treatment apparatus may be used to treat Endometriosis by application of ozone on the top of the skin above the uterus with the magnet on the opposite side. The ozone therapeutic treatment apparatus may be used to treat Hypothyroidism by application of ozone to the top of the location of the thyroid with the magnet behind it.

The ozone therapeutic treatment apparatus may be used to treat respiratory associated conditions or disease or infections. Application and treatment of the head and/or lungs may improve allergies. Asthma may be treated by the application of ozone to the lungs and directing the ozone using magnets. Emphysema, or other lung defects, with ozone introduced intravenously, and then directed affected location with a magnet to pull it through and saturate the area.

Spider veins and varicose veins may be treated with ozone therapeutic treatment apparatus by the introduction of ozone into or proximal to the veins, such as in the tissue around the vein and the application of magnets on the surface of the skin over the veins. Also, a magnetic ozone interface may be used to introduce ozone through a magnet or pull ozone from the tissue proximal the veins.

The ozone therapeutic treatment apparatus may be used to treat infections, either internally or external infections, such as on the skin, or in the eye, nose, mouth or ears. Gangrene may be treated with the ozone therapeutic treatment apparatus, for example. A magnetic ozone interface may be used to introduce ozone through a magnet or pull ozone from the tissue proximal the veins. Also, septicemia, blood poisoning from bacteria, may be treated by injection of ozone into the venous system and movement of the ozone using the magnets.

The ozone therapeutic treatment apparatus may be used to treat join related injuries and conditions by injection of ozone into the joint and moving the ozone across the joint with the magnet or magnets.

The ozone therapeutic treatment apparatus may be used to sterilize an animal-based food product, meats used as food products, such as poultry, fowl, pigs, lamb, cows, etc. The meat may be placed in an ozone cup and a magnet may be placed on the other side of the meat to promote the flow of ozone into the meat for sterilization purposes.

The ozone may be incorporated into a liquid that is configured on the patient's skin and the ozone may be small bubbles, or even nano-bubbles having a size of a few microns or even a micron or less.

The ozone therapeutic treatment apparatus may be used to create ozone gels with higher concentrations of ozone. An ozone gel may be olive oil that is infused with ozone and using a magnet to draw the ozone into the olive oil may increase the concentration or saturation of ozone in the olive oil gel. Other liquids may be used to produce an ozone gel.

A sound device, such as an ultrasound device for producing ultrasound may be used in combination with one or more of the ozone introduction techniques described herein. The sound and pulsating sound may increase absorption of ozone into the skin.

An electrical device may be used to impart or introduce DC current or DC pulsed current in combination with one or more of the ozone introduction techniques described herein. Also, a Transcutaneous Electrical Nerve Stimulation (TENS) device may be used with one or more of the ozone introduction techniques described herein. A TENS device uses low-voltage electrical currents to stimulate muscle contraction and increase blood flow. A TENS unit is may be used for relieving pain by delivering current at or near a person's nerves to block pain signals or change said person's perception of pain. In addition, electrical frequency may be used to charge the ozone as it goes through the cells. The electrical frequency may help maintain the ozone charge as it goes through the body.

Likewise, sound waves as well as pulsed sound waves, vibration therapy, shock wave therapy, compression and pulsed compression (similar to DVT products) Deep vein thrombosis (DVT) radio frequencies, pulsed lasers and the like may be used in combination with one or more of the ozone introduction techniques described herein and may stimulate tissue and/or increase blood flow to a treatment location. A Deep vein thrombosis (DVT) type device may be used to apply compression to a body part, such as around a portion of a person's leg or arm.

The north side of a magnet may be configured on the patient's skin closest to the treatment site.

The ozone therapeutic treatment apparatus may also be used to introduce oxygen or a mixture of oxygen and ozone into a person's body, such as through absorption through the skin, or by injection. Pure oxygen or oxygen with some other gas may be introduced through the methods and using the apparatus as described herein. The concentration of oxygen may be pure or substantially 100% oxygen, such as at least 95% oxygen or more, about 75% oxygen or more, about 50% oxygen or more, about 40% oxygen or more and any range between and including the concentration percentages provided. Some other gases that may be combined with the oxygen include nitrogen, helium, carbon dioxide, argon, ozone and any mixture thereof. The concentration of oxygen to ozone may that may be introduced may be 0.1:0.9 or more, about 0.25:0.75 or more, about 0.5:0.5 or more, about 0.75:0.25 or more, about 0.9:0.1 or more, about 0.95:0.05 or more, and any range between and including the ratios provided, which may be ratios or partial pressure of the two gases, wherein the sum of the mole fractions of all the components present must equal 1. The partial pressure of any gas in a mixture is the total pressure multiplied by the mole fraction of that gas. This conclusion is a direct result of the ideal gas law, which assumes that all gas particles behave ideally. A mixture of oxygen and ozone, or some other gas or mixture of gasses may further aid in healing as oxygen may aid in oxygenation of blood and tissue.

A patient, as used herein, is an example of a living body. A living body may be a person, an animal such as a dog, cat, horse, cow, and the like.

The invention provides a method of treating a treatment location or condition using the ozone therapeutic treatment apparatus as described in any of the embodiments herein. The method includes introducing ozone into the living body through any means as described herein and then directing or moving the ozone to the treatment location via a magnetic field, wherein a magnet field is introduced into the living body by a magnet, such as a magnet being placed on the skin or outer surface of the living body. A magnet may be placed on an opposing side of the living body from the location of introduction of the ozone into the living body. For example, ozone may be introduced on one side of the torso, the chest, and the magnet may be placed on the back of the torso, the living body back to direct the ozone through the torso of the living body. Likewise, ozone may be introduced on a first side of joint and the magnet may be placed on an opposing side of the joint to direct the ozone into the joint. The ozone may be introduced in to a first side of a limb, leg, arm of a living body and the magnet may be place on an opposing side from said first side. The ozone may be introduced in to a first side of a torso, head, abdomen, appendage of a living body and the magnet may be place on an opposing side from said first side.

The summary of the invention is provided as a general introduction to some of the embodiments of the invention, and is not intended to be limiting. Additional example embodiments including variations and alternative configurations of the invention are provided herein.

### BRIEF DESCRIPTION OF SEVERAL VIEWS OF THE DRAWINGS

The accompanying drawings are included to provide a further understanding of the invention and are incorporated in and constitute a part of this specification, illustrate embodiments of the invention, and together with the description serve to explain the principles of the invention.

FIG. 1 shows a perspective view of an exemplary ozone therapeutic treatment apparatus having a plurality of ozone introducers, a magnetic ozone interface, and an ozone injector to implant ozone into the body through the skin, and a second magnetic ozone interface that produces a magnetic field opposite the first magnetic ozone interface to draw the ozone through the body toward the second magnetic ozone interface.

FIG. 2 shows a perspective view of an exemplary ozone therapeutic treatment apparatus being used to treat scar tissue in a shoulder joint.

FIG. 3 shows a perspective view of an exemplary ozone therapeutic treatment apparatus being used to treat tumor in the neck of a patient.

FIG. 4 shows a perspective view of an exemplary ozone therapeutic treatment apparatus being used to a virus in a genitals of a patient.

FIG. 5 shows a chart of magnetic strength as a function of air gap distance from the magnet.

FIG. 6 shows a perspective view of a sauna treatment apparatus having an enclosure and a head opening.

FIG. 7 shows a perspective view of a person having blood drawn treated by an ozonating blood apparatus to add ozone to the blood and produce ozonated blood that may be then returned to the person.

FIG. 8 shows a person having ozonated blood being injected into a treatment location, their shoulder and a magnetic ozone interface configured to direct the ozonated blood to the treatment location.

FIG. 9 shows a magnetic ozone interface that includes a magnet that is porous to allow for ozone gas to flow through the magnet and a pressure apparatus to produce a flow of ozone through the magnet.

FIG. 10 shows a magnetic ozone cup that includes a light emitter to emit light onto the patient's skin.

FIG. 11 shows a side view of a food product being sterilized using an exemplary ozone therapeutic treatment apparatus.

FIG. 12 shows a side view of an exemplary magnetic ozone interface having a ring magnet coupled to an ozone cup assembly having an internal ozone cup surrounded by a collection cup.

FIG. 13 shows a bottom view of an exemplary ring magnet having a center aperture surrounded by a magnet.

FIG. 14 shows a perspective view of an exemplary magnetic ozone interface wrapped around a person limb, their leg and comprising an internal ozone cup and collection cup configured around the collection cup, and a magnet configured over a treatment location.

FIG. 15 shows a perspective view of an exemplary magnetic ozone interface configured on a person's head with introduction of the ozone into the internal cup of the internal cup assembly and withdrawn by the collection cup.

FIG. 16 shows a perspective view of an exemplary magnetic ozone interface configured on a person's leg with introduction of the ozone into the internal cup of the internal cup assembly and withdrawn by the collection cup as well as ozone being injected and magnets on opposite sides of the leg.

Corresponding reference characters indicate corresponding parts throughout the several views of the figures. The figures represent an illustration of some of the embodiments of the present invention and are not to be construed as limiting the scope of the invention in any manner. Some of the figures may not show all of the features and components of the invention for ease of illustration, but it is to be understood that where possible, features and components from one figure may be included in the other figures. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention.

### DETAILED DESCRIPTION OF THE ILLUSTRATED EMBODIMENTS

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Certain exemplary embodiments of the present invention are described herein and are illustrated in the accompanying figures. The embodiments described are only for purposes of illustrating the present invention and should not be interpreted as limiting the scope of the invention. Other embodiments of the invention, and certain modifications, combinations and improvements of the described embodiments, will occur to those skilled in the art and all such alternate embodiments, combinations, modifications, improvements are within the scope of the present invention.

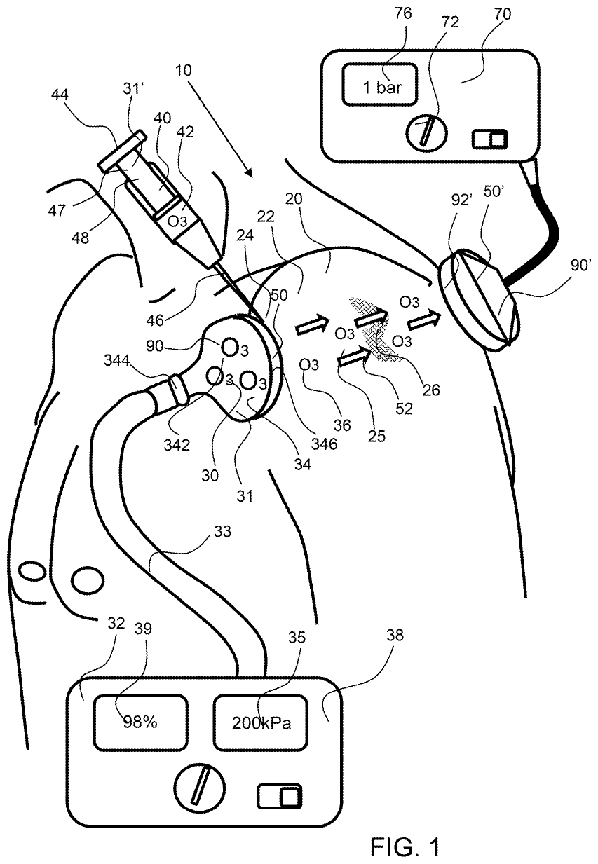

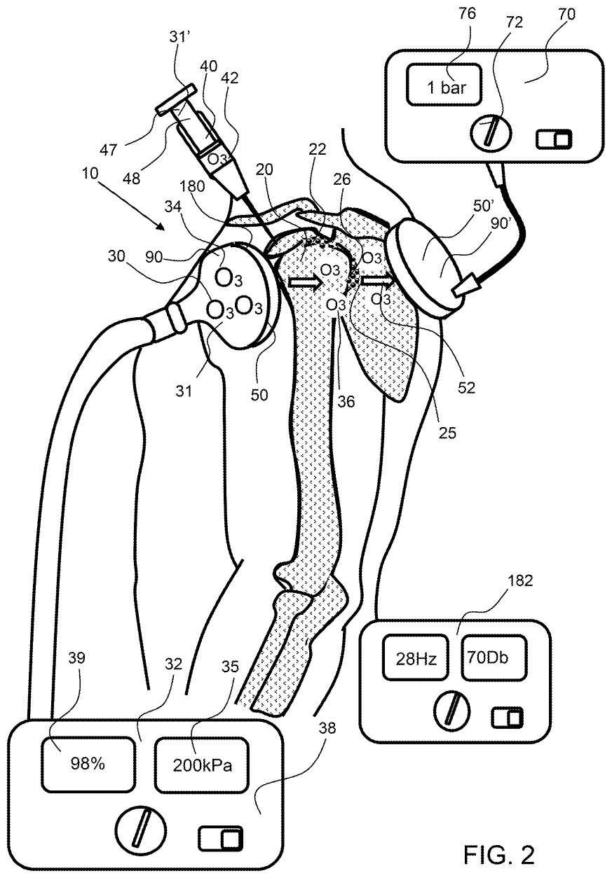

Referring now to FIGS. 1 and 2, an exemplary ozone therapeutic treatment apparatus 10 has a plurality of ozone introducers 31, 31′, a magnetic ozone interface 90 having an ozone cup 34 and a magnet 50, and an ozone injector 40 to implant ozone into the body 20 through the skin 22. A second magnetic ozone interface 90′ has a second magnet 50′ which may be a porous magnet 92 that allows gas to flow through the magnet. The first magnet 50 may produce a magnetic field that forces or drives the ozone into the body and the second magnet 50′, such as a porous magnet 92′ may produce a magnetic field 52 to draw the ozone 30 and the internal ozone 36, the ozone implanted in the body, through the body toward the second magnetic ozone interface 90′. The internal ozone 36 moves from the ozone introducer(s) or implanted area toward the second magnetic ozone interface 90′ across a treatment condition 25, scar tissue 26 of the shoulder joint. Ozone is known as an effective treatment for breaking up scar tissue and relieving the symptoms of frozen shoulder and actively moving the ozone across and or through the joint via the magnetic field enhances the effectiveness of this treatment.

The ozone cup 34 of the first magnetic ozone interface 90 has an inlet end 344 that is coupled with an ozone source 32, such as an ozone generator 38, which may be used to control the ozone concentration 39 and/or the ozone pressure 35 supplied to the ozone introducer 31. The ozone cup 34 has an interior volume for the received ozone 30 and an application end 346 configured to dispense the ozone to a treatment location 24 through an opening in the application end. Note that the ozone generator may be coupled with the ozone injector 40 as well to provide a flow of ozone into the body through the needle 46. The ozone injector 40 has an internal receptacle 42 for retaining or receiving ozone 30 and a plunger 44 to force the ozone through the needle and into the body 20. The ozone injector 40 may be a jet injector 47 that utilizes a pressurizing device 48 to introduce the ozone 30 into the living body under high pressure.

The magnets 50, 50′ may be a permanent magnet or an electromagnet that is powered by an electromagnet generator that may enable control of the frequency and amplitude of the magnetic field. The magnets may be porous magnets to allow for the flow of gas, such as ozone therethrough.

Note that the ozone introducer(s) 31 is configured on a first side of the treatment condition 25, or shoulder joint and the magnet 50′ of the second magnetic ozone interface 90′ is configured on the other side of the treatment condition or shoulder joint, to enable the implanted ozone, the internal ozone 36 to pass through the treatment condition. Also, note that the second magnetic ozone interface 90′ is coupled with a pressure apparatus 70 to produce a vacuum of 1 bar on the skin surface to further promote flow of gases through the body and across the treatment location.

A pressure wave apparatus 180 may be coupled with an ozone introducer to apply a pressure wave, such as sound waves to the person's body, which may increase blood flow and may aid to force ozone into the body. A pressure wave controller 182 may be used to control the frequency and pressure applied.

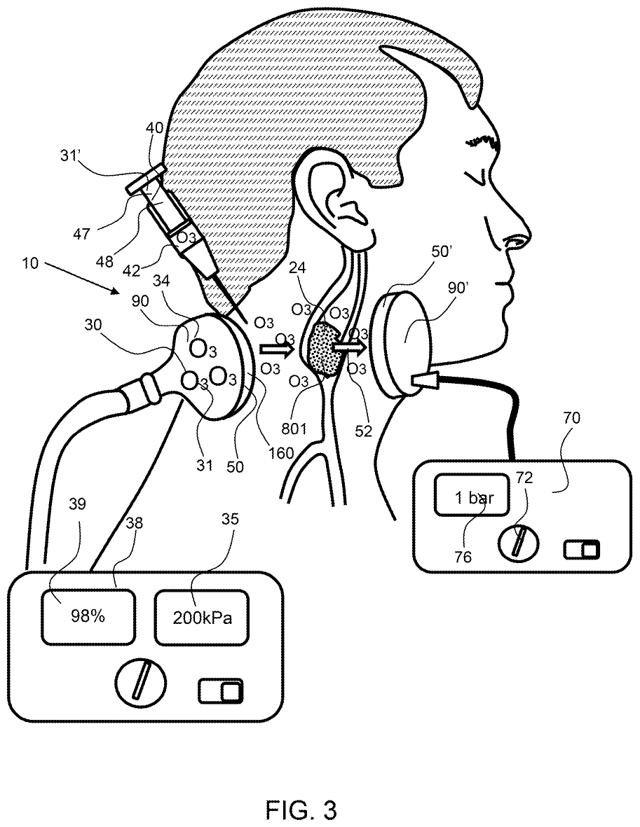

As shown in FIG. 3, an exemplary ozone therapeutic treatment apparatus 10 is being used to treat a tumor 801 in the neck of a patient. As shown in the FIGS. 1 and 2, the ozone cup 34 is coupled with an ozone source 32, such as an ozone generator 38, which may be used to control the ozone concentration 39 and/or the ozone pressure 35 supplied to the ozone introducer 31. Note that the ozone generator may be coupled with the ozone injector 40 as well to provide a flow of ozone into the body through the needle 46. The ozone injector 40 has an internal receptacle 42 for retaining or receiving ozone 30 and a plunger 44 to force the ozone through the needle and into the body 20. In addition, the magnet 50 may be a permanent magnet or an electromagnet that is powered by an electromagnet generator that may enable control of the frequency and amplitude of the magnetic field 52. Note that the ozone introducers 31, 31′ are configured on a first side of the treatment condition 25, or shoulder joint and the magnet is configured on the other side of the treatment condition or shoulder joint, to enable the implanted ozone, the internal ozone 36 to pass through the treatment condition. Again, a first magnetic ozone interface 90 and second magnetic ozone interface 90′ may be configured on opposing sides of the treatment location 24 to create a push pull of ozone through the treatment location. A Transcutaneous Electrical Nerve Stimulation (TENS) device 160 may be configured with the ozone therapeutic treatment apparatus and may apply electrical current to the person to stimulate blood flow and/or cause muscle contraction to aid in ozone absorption. Note that the TENS device may provide DC current, pulsed DC current and alternating voltages to increase blood flow for more effective ozone absorption.

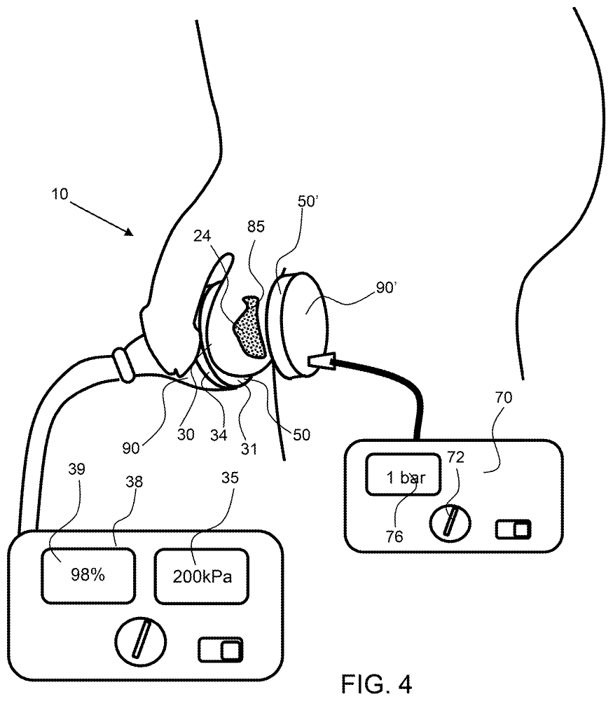

FIG. 4 shows a perspective view of an exemplary ozone therapeutic treatment apparatus being used to treat a virus in a genitals of a patient. Again, a first magnetic ozone interface 90 and second magnetic ozone interface 90′ may be configured on opposing sides of the treatment location 24 to create a push pull of ozone through the treatment location.

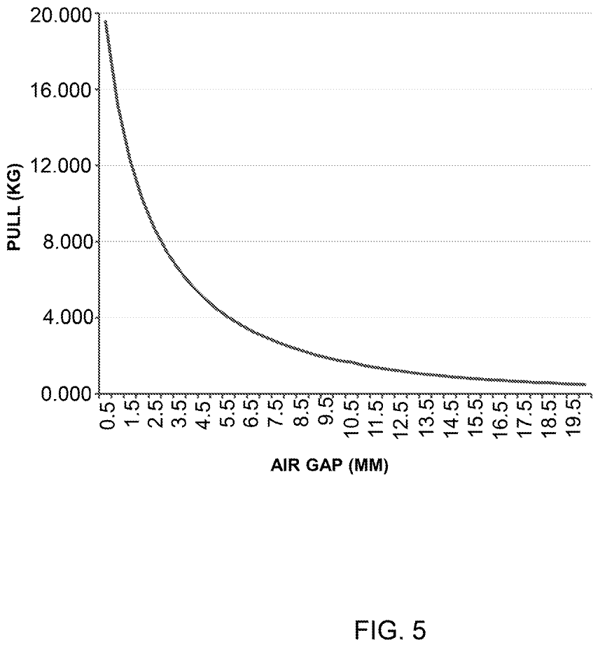

As shown in FIG. 5, the magnetic field strength diminishes quickly with the increasing distance from the magnet. Therefore, placing the magnet on or very near the skin may be desirable for the treatment apparatus.

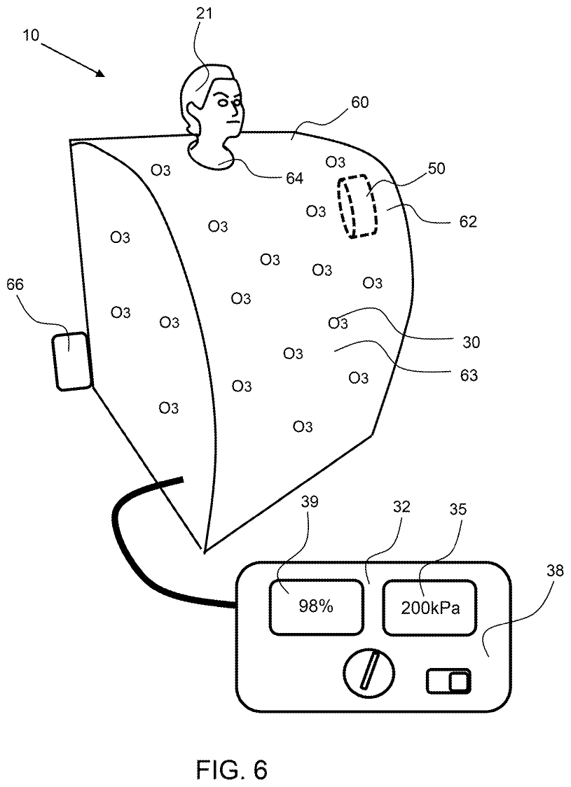

As shown in FIG. 6, a heated sauna 60 forms and enclosure 62 for retaining ozone 30 against a body part. The interior 62 of the sauna is heated by a heating element 66. A head opening 64 in the enclosure enables the patient 21 (living body) to extend their head out of the enclosure. Ozone 30 is configured in the interior of the enclosure and a magnet 50 may be placed against the persons skin to draw ozone into the person's body and across a treatment condition. An ozone generator 38 is the ozone source 32 for providing a flow of ozone 30 into the interior 63 of the enclosure 62. Ozone may be introduced with steam or heated water vapor and the interior of the chamber may be pressurized to increase absorption of ozone. Steam may be initially introduced with the ozone but may cool to be heated water vapor upon contact with the patient to avoid burning due to excessive heat.

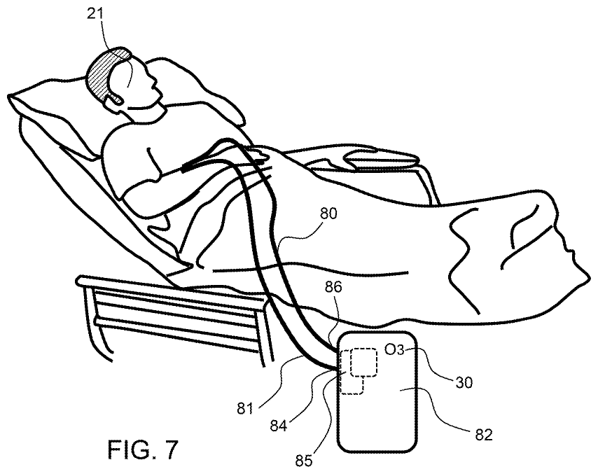

As shown in FIG. 7, a patient 21 is having blood drawn and treated by an ozonating blood apparatus 82 to add ozone 30 to the blood 81 and produce ozonated blood 80 that may be then returned to the patient. As described herein, this may be used to treat any blood born viruses or infections and may be used to treat autoimmune diseases. The ozonating blood apparatus 82 has an inlet 84 for receiving the blood 81 and an ozonated blood outlet 86 to deliver the ozonated blood 80 back to the patient. A pump 85 may control the flow of blood from and/or to the person.

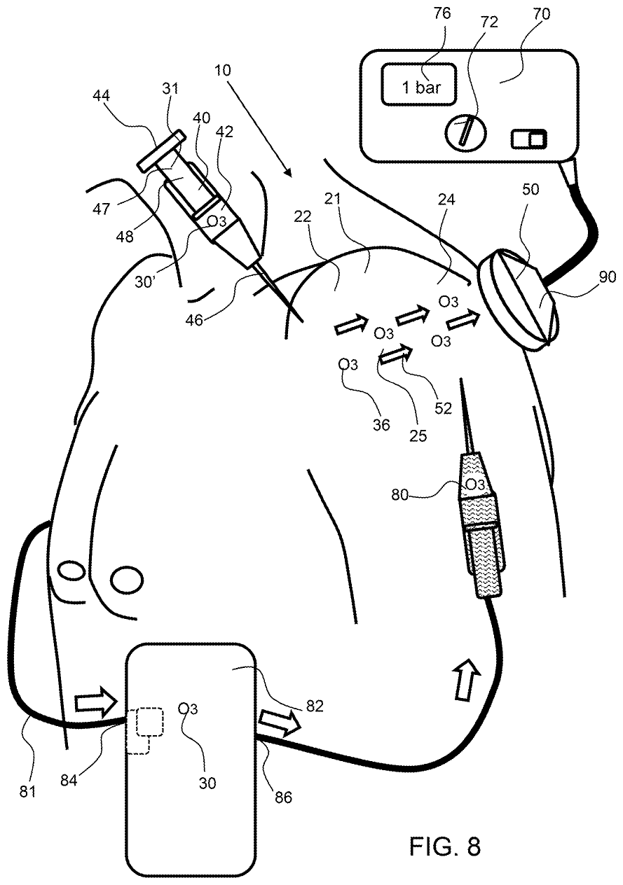

As shown in FIG. 8, a person is having ozonated blood 80 injected into a treatment location 24, their shoulder and a magnetic ozone interface 90 configured to direct the ozonated blood 80 to the treatment location. An ozonating blood apparatus 82 is drawing blood 81 from a patient 21, adding ozone 30 to the blood to produce ozonated blood 80 that is being injected into the patient's shoulder, the treatment location. The ozonated blood 80 is being drawn to the treatment location by a magnetic ozone interface 90 having a porous magnet 92 to allow the flow gas therethrough and a pressure apparatus 70 configured to produce a vacuum pressure on the magnetic ozone interface 90. The pressure apparatus 70 has a pressure display 76 and a pressure controller 72 to change the pressure on the magnetic ozone interface. Also, additional ozone 30′ may be injected into or proximal to the treatment location via an introducer 31, such as an ozone injector 40, having a plunger 44 to force ozone in an internal receptacle 42 through a needle 46 and into the skin 22 and into the body.

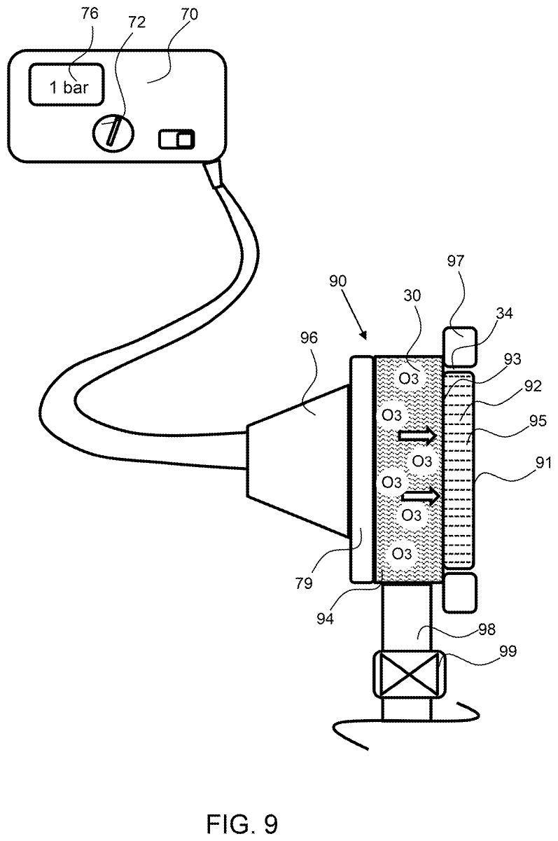

As shown in FIG. 9, a magnetic ozone interface 90 includes a porous magnet 92 having apertures 95 extending from a skin contact surface 91 to a cup surface 93 to allow for ozone 30 to flow through the magnet, and a pressure apparatus 70 to produce a flow of ozone through the magnet. The porous magnet 92 is configured within a gasket 97 that may be affixed to a patient's body to create a seal around the porous magnet. The magnetic ozone interface 90 has a gas coupling 96 to deliver or remove gas, which may include ozone 30 to the ozone reservoir 94. A magnetic ozone interface 90 may have a reservoir conduit 98 coupled to the ozone reservoir and having a reservoir conduit valve 99 to control the flow of gas to or from the ozone reservoir. In an exemplary embodiment, ozone is supplied to the ozone reservoir and therefore through the apertures 95 in the porous magnet 92 and is bled off through the reservoir conduit. A fresh stream of ozone from the pressure apparatus 70 provides a high and consistent ozone concentration in the ozone reservoir with some gas flowing out from said reservoir through the reservoir conduit.

A heating element 79 is configured with the ozone cup 34 and configured to heat the skin of the patient for more effective absorption of ozone into the patient. The heating element may be an electrically resistive heating element or a radiant heating element, a light with a wavelength configured to heat the skin.

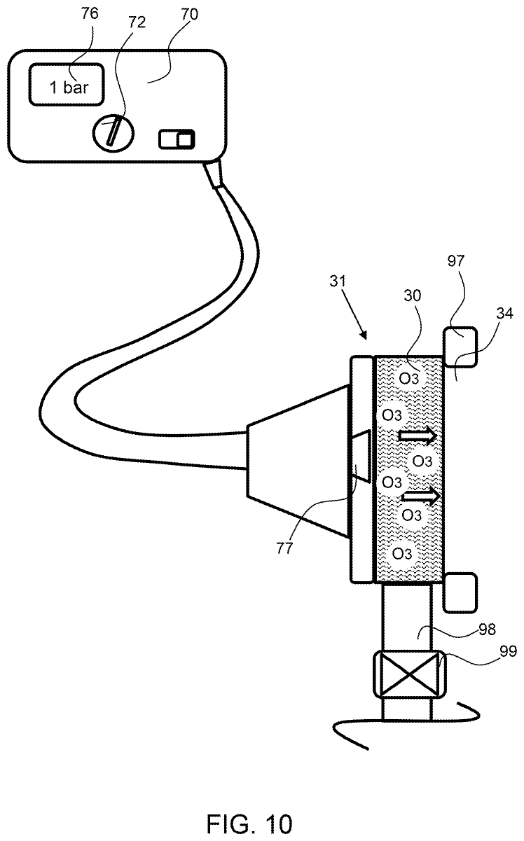

As shown in FIG. 10, an ozone introducer 31 has an ozone cup 34 with a light emitter 77 configured to emit light onto the skin of the patient and/or food product.

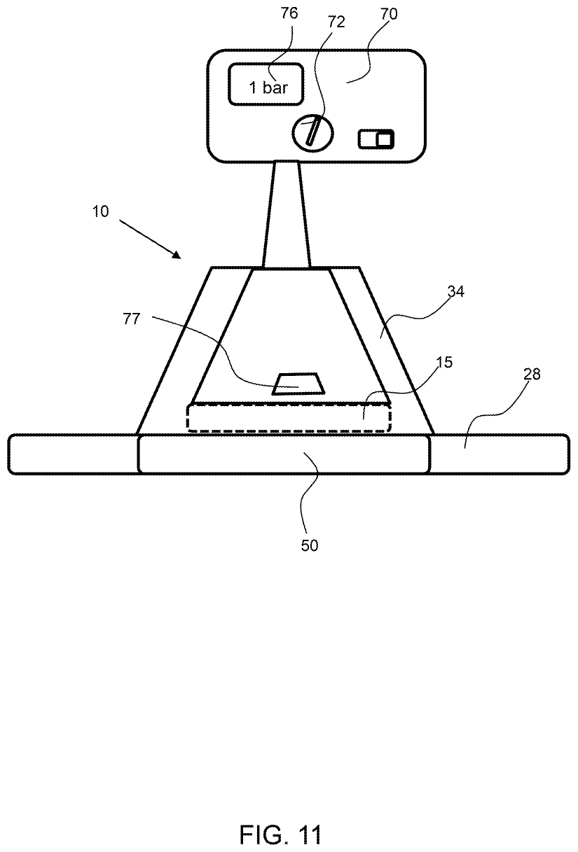

As shown in FIG. 11, an animal-based food product 15, such as a chicken breast, is being sterilized with an exemplary ozone therapeutic treatment apparatus, wherein the chicken breast is configured in an ozone cup and a magnet 50 is configured on the opposing side of the chicken breast from the ozone cup. The ozone 30 is drawn through the food product 15 to sterilize the food product. The magnet may be coupled to a conveyor belt 28 to enable high speed sterilization of the food product 15. A light emitter 77 may be configured in the ozone cup 34 to produce a light onto the food product. The light may be an ultraviolet light that is configured to disinfect the food product by destroying or inactivating pathogens.

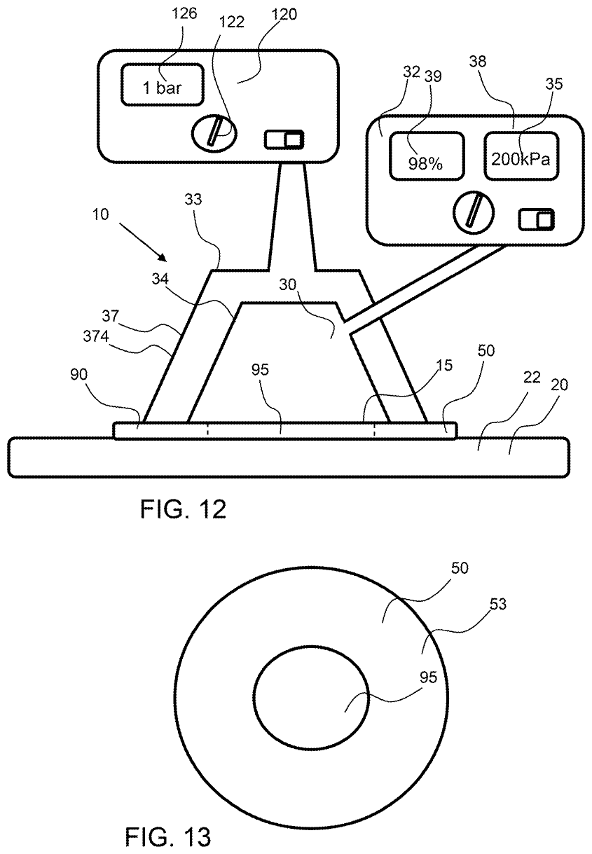

Referring now to FIGS. 12 to 14, an exemplary magnetic ozone interface 90 has a ring magnet 53 coupled to an ozone cup assembly 33 having an internal ozone cup 34 surrounded by a collection cup 37. The internal ozone cup 34 receives ozone 30 from an ozone source 32 that may force ozone into the interior of the ozone cup 34 with pressure, as indicated by the pressure gauge 35. The ozone source may be an ozone generator 38 and may produce pure ozone with a concentration as shown in the concentration gauge 39. The collection cup 37 is configured around the ozone cup and configured to capture an ozone that leaks from the interior ozone cup 34. The collection cup is coupled with an ozone destruction device 120 that may create a vacuum pressure as indicated by the pressure gauge 126 and controlled by the pressure regulator 122. The magnet 50 may be a ring magnet 53 with a center aperture 95 to allow the ozone to pass into or out of the body 20, or through the skin 22. The ozone cup assembly 33 may be attached to the magnet 50 to prevent leakage of ozone between the ozone cup assembly and magnet. The magnet may be a switchable magnet configured to turn on and off.

A collection cup 37 may include an ozone destruction material 374, that is configured to destroy the ozone, such as by reaction on a catalyst. Manganese dioxide is an ozone catalyst.

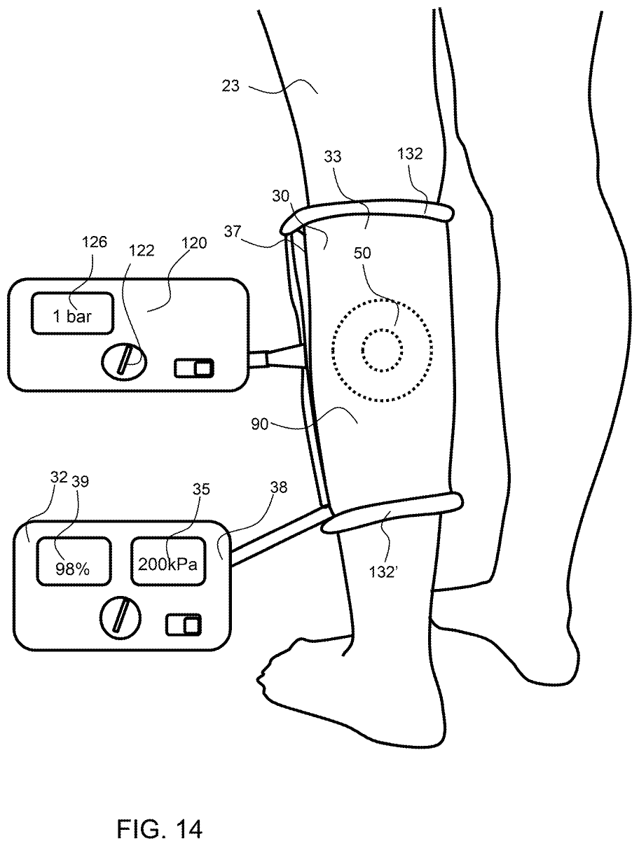

As shown in FIG. 14, an exemplary magnetic ozone interface 90 is wrapped around a person limb 23, their leg, and comprising an internal ozone cup 34 and collection cup 37 configured around the collection cup, and a magnet 50 configured over a treatment location. Again, the magnet may be a switchable magnet configured to turn on and off. As described herein, an exemplary magnetic ozone interface may be configured to wrap around a body part including over the shoulder, head, around the back or hip of a person or animal and the like. The ozone cup assembly 33 shown in FIG. 14, may be incorporated with a compression device 130, such as a DVT device that produces pressure around the limb, or compresses the limb. The compression device may include a bladder 134 that is inflated to produce compression around the limb 23 and may include cuffs 132, 132′, which may be inflated or otherwise cinched to prevent ozone 30 from escaping from the bladder 134. The pressure may be pulsed to increase blood flow to the limb.

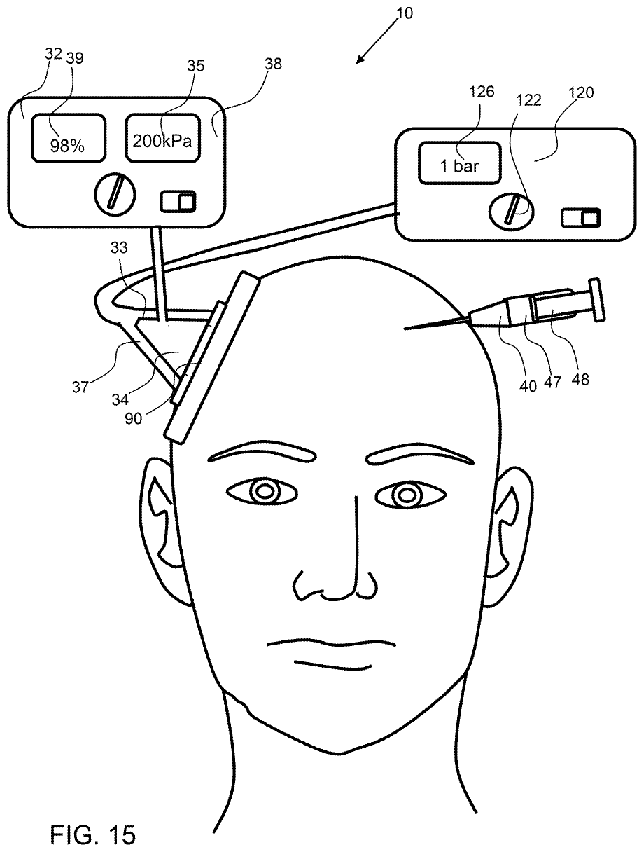

As shown in FIG. 15, an exemplary 90 magnetic ozone interface is configured on a person's head with introduction of the ozone into the internal ozone cup 34 of the ozone cup assembly 33 and withdrawn by the collection cup 37. An ozone injector 40 may be used to inject ozone into the brain as well and this injected ozone may move across the brain to the magnetic ozone interface.

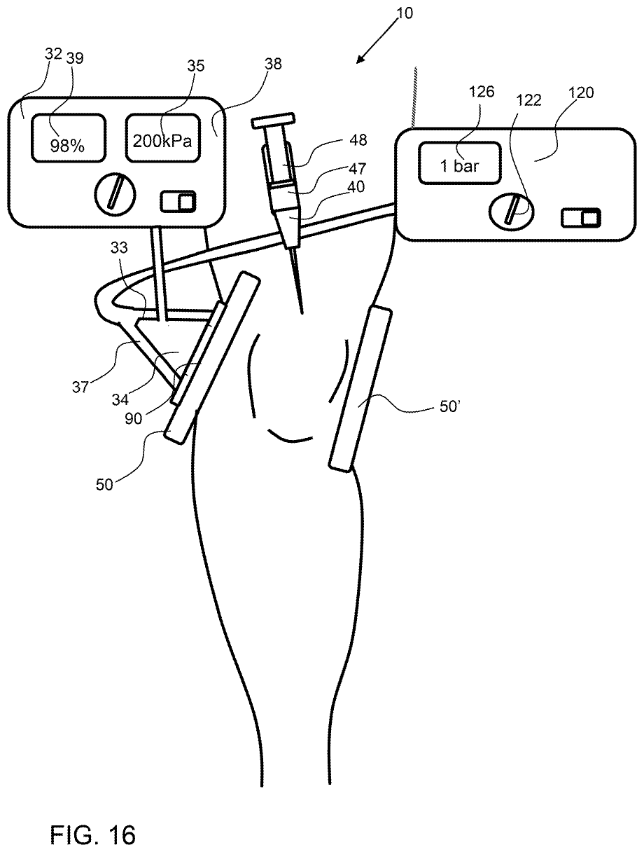

As shown in FIG. 16, an exemplary 90 magnetic ozone interface is configured on a person's leg 200 proximal to the knee 202 with introduction of the ozone into the internal ozone cup 34 of the ozone cup assembly 33 and withdrawn by the collection cup 37. An ozone injector 40 may be used to inject ozone into the tissue around the knee as well and this injected ozone may move across the leg and across the knee to the magnetic ozone interface and/or to the second magnet 50′, opposite the first magnet 50 and opposite the ozone cup 34. Note that a first magnet 50 and second magnet 50′ may be configured on the person's body, such as on opposing or opposite sides of the body or on opposite sides of where the ozone is being introduced such as by an ozone injector 34 and/or ozone cup 34.

It will be apparent to those skilled in the art that various modifications, combinations, and variations can be made in the present invention without departing from the scope of the invention. Specific embodiments, features and elements described herein may be modified, and/or combined in any suitable manner. Thus, it is intended that the present invention cover the modifications, combinations and variations of this invention provided they come within the scope of the appended claims and their equivalents.

What is claimed is:

1. An ozone therapeutic treatment apparatus comprising:
a) an ozone source;
b) an ozone introducer configured to transfer ozone into a living body;
c) a magnet that produces a magnetic field;
wherein the ozone is configured to be driven through said living body toward the magnet by the magnetic field and wherein the ozone is configured to treat a treatment location of said living body
wherein the ozone is configured for introduction into a first side of the living body and wherein the magnet is configured for placement on a second side of the living body, opposite the first side.

2. The ozone therapeutic treatment apparatus of claim 1, wherein the ozone introducer comprises an ozone cup that is configured to be placed on skin of said living body to form a seal that retains said ozone against said skin and wherein the ozone is configured to be transferred into said body through adsorption through said skin.

3. The ozone therapeutic treatment apparatus of claim 1, wherein the ozone cup introduces both ozone and heated water vapor.

4. The ozone therapeutic treatment apparatus of claim 1, wherein the ozone introducer comprises an ozone injector configured to inject ozone through the skin of the living body and into the living body.

5. The ozone therapeutic treatment apparatus of claim 4, wherein the ozone injector comprises a needle having a conduit and an aperture for injecting ozone into the body.

6. The ozone therapeutic treatment apparatus of claim 5, wherein the ozone injector comprises a syringe having an interior receptacle for retaining ozone and a plunger for pressing the ozone out of the interior receptacle through the needle and into the body.

7. The ozone therapeutic treatment apparatus of claim 4, wherein the ozone introducer injects ozone and saline through the skin of the living body and into the living body.

8. The ozone therapeutic treatment apparatus of claim 1, wherein the ozone introducer is a jet injector that utilizes a pressurizing device configured to introduce ozone into said living body under high pressure.

9. The ozone therapeutic treatment apparatus of claim 1, wherein the ozone introducer is an ozone cup having an interior volume for receiving ozone, an inlet for receiving ozone from said ozone source and an application end having an opening for dispensing ozone to said treatment location.

10. The ozone therapeutic treatment apparatus of claim 9, wherein the ozone cup is a compression device that applies pressure to said portion of said person's body.

11. The ozone therapeutic treatment apparatus of claim 10, wherein the ozone cup extends around a portion of a person's body.

12. The ozone therapeutic treatment apparatus of claim 11, wherein the ozone cup is a deep vein thrombosis device that compresses extends around a portion of a limb of said person and pressurizes said limb.

13. The ozone therapeutic treatment apparatus of claim 1, wherein the ozone introducer comprises a sauna comprising an enclosure for at least a portion of said body and wherein said enclosure contains ozone.

14. The ozone therapeutic treatment apparatus of claim 13, wherein an interior of the sauna is heated to an elevated temperature of at least 30° C.

15. The ozone therapeutic treatment apparatus of claim 13, wherein the enclosure has an ozone concentration of 50 ppm or more.

16. The ozone therapeutic treatment apparatus of claim 13, wherein the enclosure is configured for entry and enclosure of a person therein.

17. The ozone therapeutic treatment apparatus of claim 13, wherein the sauna comprises heated water vapor and ozone within the enclosure.

18. The ozone therapeutic treatment apparatus of claim 1, wherein the magnet is a permanent magnet.

19. The ozone therapeutic treatment apparatus of claim 1, wherein the magnet is an electro-magnet that produces a change in said magnetic field.

20. The ozone therapeutic treatment apparatus of claim 1, wherein the magnet is a switchable magnet.

21. The ozone therapeutic treatment apparatus of claim 1, further comprising a sound device that creates a sound wave on the skin of said living body where the ozone is introduced into said living body.

22. The ozone therapeutic treatment apparatus of claim 1, further comprising an electrical device that introduces a DC current into the skin of said living body where the ozone is introduced into said living body.

23. The ozone therapeutic treatment apparatus of claim 22, wherein the electrical device is a TENS device.

24. The ozone therapeutic treatment apparatus of claim 1, wherein the ozone is charged ozone.

25. An ozone therapeutic treatment apparatus comprising:
a) an ozone source;
b) an ozone introducer configured to transfer ozone into a living body;
c) a magnet that produces a magnetic field;
wherein the ozone is configured to be driven through said living body toward the magnet by the magnetic field and wherein the ozone is configured to treat a treatment location of said living body;
wherein the ozone introducer is an ozone cup assembly comprising:
d) an ozone cup having an interior volume for receiving ozone;
e) a collection cup configured around the ozone cup and configured to collect ozone leaked from the ozone cup;
wherein the ozone cup comprises an inlet for receiving ozone from said ozone source and an application end having an opening for dispensing ozone to said treatment location; and
wherein the collection cup is coupled with a pressure apparatus configured to create a vacuum in the collection cup to remove ozone from the collection cup.

26. The ozone therapeutic treatment apparatus of claim 25, wherein the collection cup comprises an ozone destruction material to destroy said ozone.

27. The ozone therapeutic treatment apparatus of claim 25, wherein the ozone cup assembly further comprises said magnet and wherein said magnet comprises an aperture to enable ozone to be transferred to the living body.

28. The ozone therapeutic treatment apparatus of claim 25, wherein the ozone introducer and magnet are configured in a magnet ozone interface, wherein ozone flows through the magnet.

29. The ozone therapeutic treatment apparatus of claim 25, wherein the ozone cup assembly comprises a second magnet and wherein said second magnet comprises an aperture to enable ozone to be transferred to the living body.

30. The ozone therapeutic treatment apparatus of claim 29, wherein the ozone introducer and magnet are configured in a magnet ozone interface, wherein ozone flows through the magnet.
